(19) United States
(12) Patent Application Publication
(10) Pub. No.: US 2005/0049464 A1
(43) Pub. Date: Mar. 3, 2005

(54) Remote pharmaceutical administration device

(76) Inventors: Harold Aaron Lassers, Elmhurst, IL (US); Ronald Bruce Martin, Carol Stream, IL (US)

Correspondence Address: Lucent Technologies Inc.;Docket Administrator (Room 3J-219), 101 Crawfords Corner Road, Holmdel, NJ, 07733-3030, US

(21) Appl. No.: 10/651,646

(22) Filed: Aug. 29, 2003

(51) Int. Cl. A61B 5/00; A61M 5/00; A61M 16/00

(52) U.S. Cl. 600/300; 604/66; 128/204.23

(57) ABSTRACT

The present invention provides a wireless communication system that includes a remote pharmaceutical administration device and a pharmaceutical network administration apparatus. The remote pharmaceutical administration device includes a monitoring device for monitoring the vital signs of a patient, an analysis processor for receiving the vital signs, a transceiver for transmitting the vital signs over a network and receiving an action plan over the network, and administration means for administering a prescribed medication to the patient. The remote pharmaceutical administration device can also include pre-stored pharmaceuticals and a remotely-run testing apparatus, such as an EKG machine, a blood sugar monitor, or a sphygmomanometer. The pharmaceutical network administration apparatus includes a network transceiver for receiving an action plan over the wireless network and a network processor that develops instructions. The network transceiver transmits the instructions over the wireless network.

### FIELD OF THE INVENTION

The present invention relates generally to wireless communication systems, and more particularly to a wireless communication system that is capable of remotely administering a pharmaceutical.

### BACKGROUND OF THE INVENTION

Patients who need medical attention typically are connected to monitoring devices. The monitoring devices obtain medical information from a patient, which is used in diagnosing the patient.

Once a diagnosis has been made, an appropriate course of action can be taken. This can include continuing current testing, performing additional testing, requesting a consultation from a specialist, or providing medication.

One problem with current testing and diagnostic practices is that the patient must be in a facility that is staffed by at least one medical professional. If the nurse or doctor is unavailable, such as when multiple patients need attention at the same time, a patient can be without needed medical attention.

An additional problem is that it is difficult for a specialist who is not located at the facility to be able to make an accurate diagnosis or prescription without the necessary information from the patient.

Therefore, a need exists for a method and system that allows for a patient to be diagnosed and treated without requiring all medical personnel to be physically present with the patient.

### BRIEF SUMMARY OF THE INVENTION

The present invention provides a wireless communication system that includes a remote pharmaceutical administration device and a pharmaceutical network administration apparatus. The remote pharmaceutical administration device is coupled to a patient and is connected to a wireless communication network. The wireless communication network is coupled to a pharmaceutical network administration apparatus. The pharmaceutical network administration apparatus gives a doctor or other medical personnel the ability to monitor the patient, prescribe medication to the patient, or perform additional tests on the patient.

A monitoring device monitors at least one vital sign of a patient. The monitoring can include determining the pulse rate, temperature, or respiratory rate of the patient. A processor processes the vital signs to determine an action plan for the patient.

The action plan, as well as the vital signs, are sent via a wireless communication network to a pharmaceutical network administration apparatus, which is preferably being monitored by a medical professional, such as a doctor. The doctor, with the assistance of the vital signs, action plan, and analysis processor, makes a determination about a medical protocol for the patient. The medical protocol is input into the pharmaceutical network administration apparatus via input means, such as a keyboard. The analysis processor processes the instructions, and if appropriate, sends the medical protocol to the remote pharmaceutical administration device via a wireless communication network.

The processor at the remote pharmaceutical administration device then determines, based upon the medical protocol received from the pharmaceutical network administration apparatus, if medication is needed by the patient. If so, the remote pharmaceutical administration device administers the prescribed medication via administration means located in proximity to the patient.

### BRIEF DESCRIPTION OF THE SEVERAL VIEWS OF THE DRAWINGS

FIG. 1 depicts a remote pharmaceutical administration device and pharmaceutical network administration apparatus coupled via a wireless communication network in accordance with an exemplary embodiment of the present invention.

FIG. 2 depicts a remote pharmaceutical administration device in accordance with an exemplary embodiment of the present invention.

FIG. 3 depicts a pharmaceutical network administration apparatus in accordance with an exemplary embodiment of the present invention.

FIG. 4 depicts a flow chart of a method for administering a prescribed medication in accordance with an exemplary embodiment of the present invention.

FIG. 5 depicts a flow chart of a method for sending a medical protocol to the remote pharmaceutical administration device in accordance with an exemplary embodiment of the present invention.

### DETAILED DESCRIPTION OF THE INVENTION

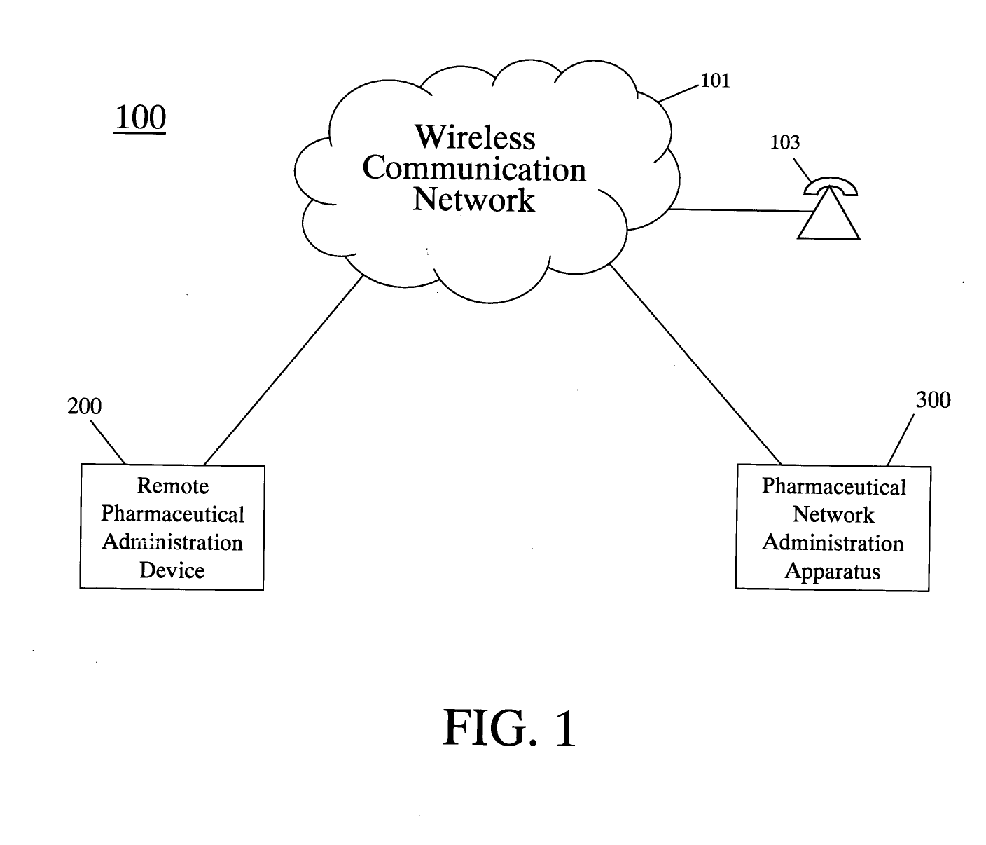

The present invention can be better understood with reference to FIGS. 1 through 5. FIG. 1 depicts a communication system 100 that includes a wireless communication network 101, a remote pharmaceutical administration device 200 and a pharmaceutical network administration apparatus 300 in accordance with an exemplary embodiment of the present invention. As described in greater detail below, remote pharmaceutical administration device 200 is coupled to a patient. Remote pharmaceutical administration device 200 is connected to wireless communication network 101, which is in turn coupled to pharmaceutical network administration apparatus 300. Pharmaceutical network administration apparatus 300 gives a doctor or other medical personnel the ability to monitor the patient, prescribe medication to the patient, or perform additional tests on the patient.

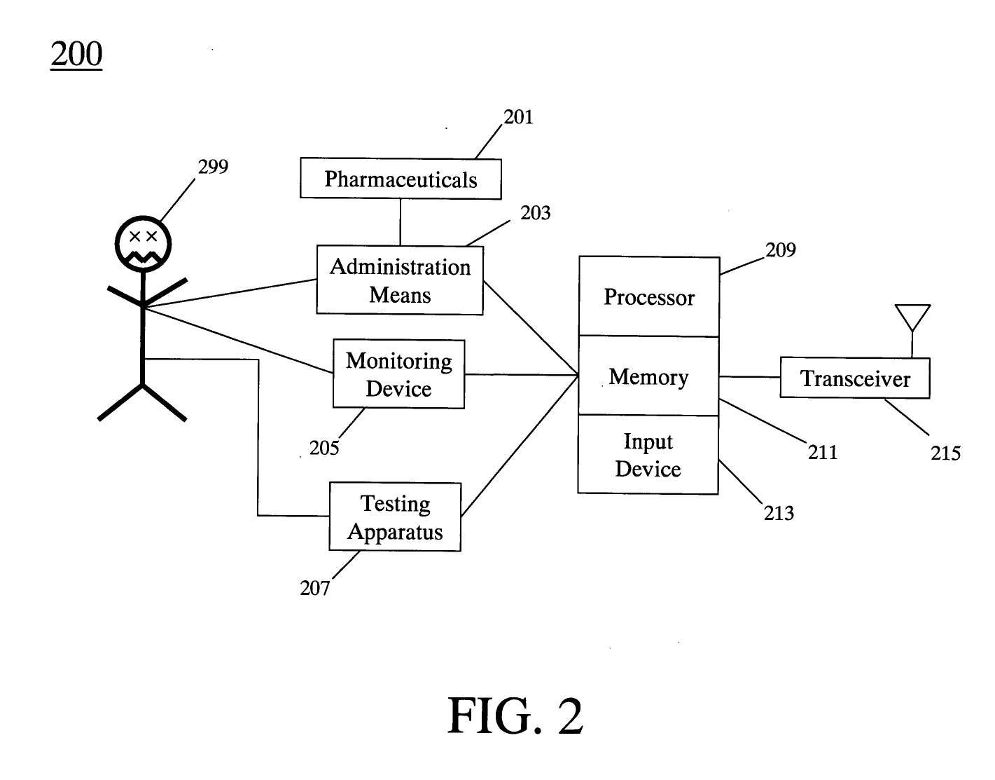

FIG. 2 depicts remote pharmaceutical administration device 200 in accordance with an exemplary embodiment of the present invention. Remote pharmaceutical administration device 200 comprises pharmaceuticals 201, administration means 203, monitoring means 205, testing apparatus 207, processor 209, memory 211, input device 213, and transceiver 215.

Pharmaceuticals 201 are preferably pre-stored medications that can be administered to patient 299. As an example, pharmaceuticals 201 may comprise oral medications, creams, inhalants, intravenous medications, subcutaneous medications, or any other type of medication.

Administration means 203 administer a prescribed medication to patient 299 based at least in part upon the action plan. Administration means 203 may be a needle, a gas dispenser such as a mask, or an apparatus for dispensing a pill or other oral medication. For example, administration means could comprise a needle that is connected to an intravenous (IV) needle or tube inserted into a vein, thereby allowing immediate access to the blood supply. Medication would be administered to the patient through the needle, into the IV needle or tube and into the patient's vein.

In a further exemplary embodiment, administration means 203 can be a mask placed over the mouth and nose of the patient that allows administration of inhalational agents in gaseous form to the patient.

Monitoring apparatus 205 monitors the vital signs of a patient. As an example, monitoring apparatus 205 can include a pulse rate monitor, a thermometer, or a respiratory rate monitor. A pulse rate monitor determines the number of heart beats in a given time period, typically by monitoring the pulse of the patient and measured in number of beats per minute.

Testing apparatus 207 is preferably a remotely-run testing apparatus. In an exemplary embodiment of the present invention, testing apparatus 207 is an Electro-Cardio-Gram, also known as an ECG or EKG. In a further exemplary embodiment, testing apparatus 207 is a blood sugar monitor. In a further exemplary embodiment, testing apparatus 207 comprises a sphygmomanometer, which is used to determine blood pressure. A sphygmomanometer comprises a cuff that can be filled with air, a hollow rubber bulb that pumps the air, and a glass tube containing a column of mercury.

In an exemplary embodiment, processor 209, memory 211, and input device 213 are embodied in a personal computer.

Processor 209 is coupled to monitoring device 205 for receiving the vital signs from monitoring device 205. Processor 209 is preferably a microprocessor embodied in a personal computer. Processor 209 is programmed with instructions to make a local medical protocol change.

Memory 211 is effective in storing data and information, and can be any storage medium capable of performing data storage and retrieval.

Input device 213 allows instructions to be entered and sent to processor 209. Input device 213 is preferably a keyboard, but can alternately be a mouse, a touch screen, voice recognition, or any other apparatus that allows input to be accepted.

Transceiver 215 is coupled to analysis processor 209 for transmitting the vital signs and receiving an action plan for the patient based at least in part upon the vital signs. Transceiver 215 can utilize any communication technique, such as CDMA (Code Division Multiple Access), IMS (IP Multimedia Subsystem), satellite-based communication protocols (such as Iridium), or CBPD.

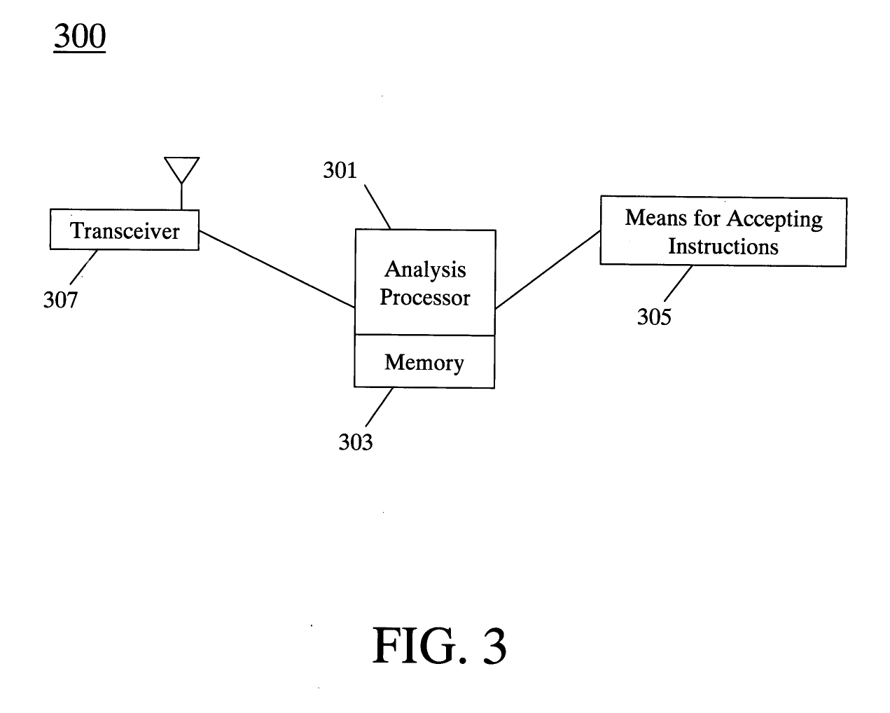

FIG. 3 depicts pharmaceutical network administration apparatus 300 in accordance with an exemplary embodiment of the present invention. Pharmaceutical network administration apparatus 300 preferably comprises an analysis processor 301, memory 303, means for accepting instructions 305, and transceiver 307.

Analysis processor 301 performs processing for pharmaceutical network administration apparatus 300. In an exemplary embodiment of the present invention, analysis processor 301 analyzes vital signs and the action plan sent from remote pharmaceutical administration device 200. Analysis processor 301 also preferably recommends protocol changes, alerts humans, and initiates replenishment of remote pharmaceuticals.

Memory 303 is effective in storing data and information, and can be any storage medium capable of performing data storage and retrieval.

Means for accepting instructions 305 allows instructions to be entered and sent to analysis processor 301. Means for accepting instructions 305 is preferably a keyboard, but can alternately be a mouse, a touch screen, voice recognition, or any other apparatus that allows input to be accepted.

Transceiver 307 transmits and receives instructions to remote pharmaceutical administration device 200 via wireless communication network 101.

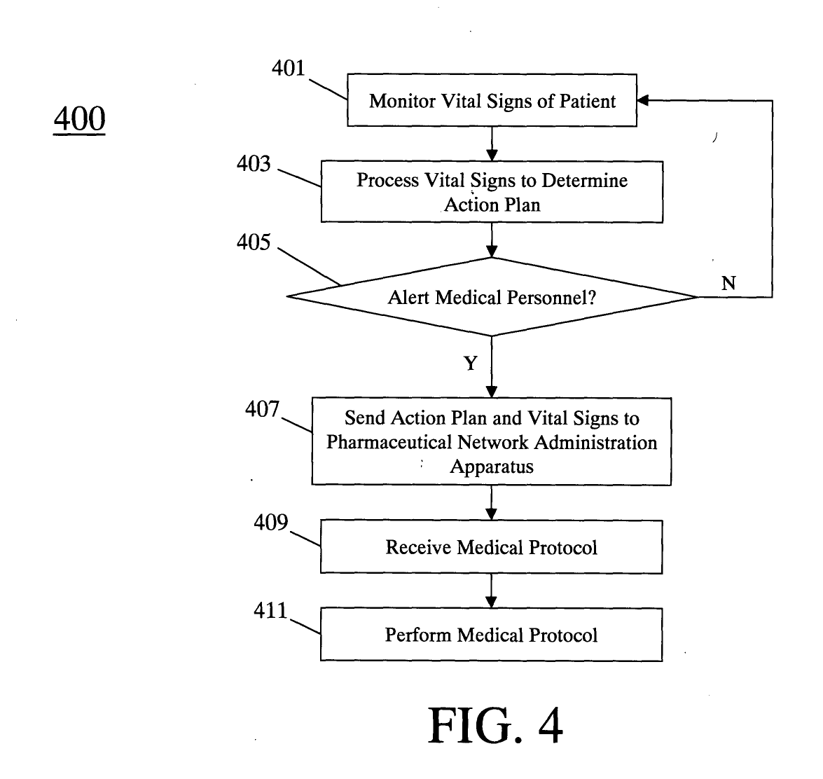

FIG. 4 depicts a flow chart 400 of a method for administering a prescribed medication by remote pharmaceutical administration device 200 in accordance with an exemplary embodiment of the present invention.

Monitoring device 205 monitors (401) a vital sign of patient 299. The monitoring can include determining the pulse rate, temperature, or respiratory rate of the patient.

Processor 209 processes (403) the vital signs to determine an action plan for patient 299. The action plan can be a generic plan used for all patients, or can be a custom action plan designed for each individual patient. For example, a generic action plan can be to alert a medical professional for any person whose pulse rate either drops below a predetermined threshold or exceeds a predetermined threshold. In an alternate exemplary embodiment, a generic action plan could be to alert a medical professional when any patient's temperature or respiratory rate falls outside of a predetermined and acceptable range.

Processor 209 determines (405) if a medical specialist, such as a doctor, should be alerted. If a medical specialist does not need to be alerted, processing returns to step 401 and the vital signs of the patient continue to be monitored.

If a medical specialist should be alerted, remote pharmaceutical administration device 200 sends (407) the action plan and vital signs to pharmaceutical network administration apparatus 300 via wireless communication network 101 utilizing transceiver 215. The action plan can also include the patient's medical history and medical file.

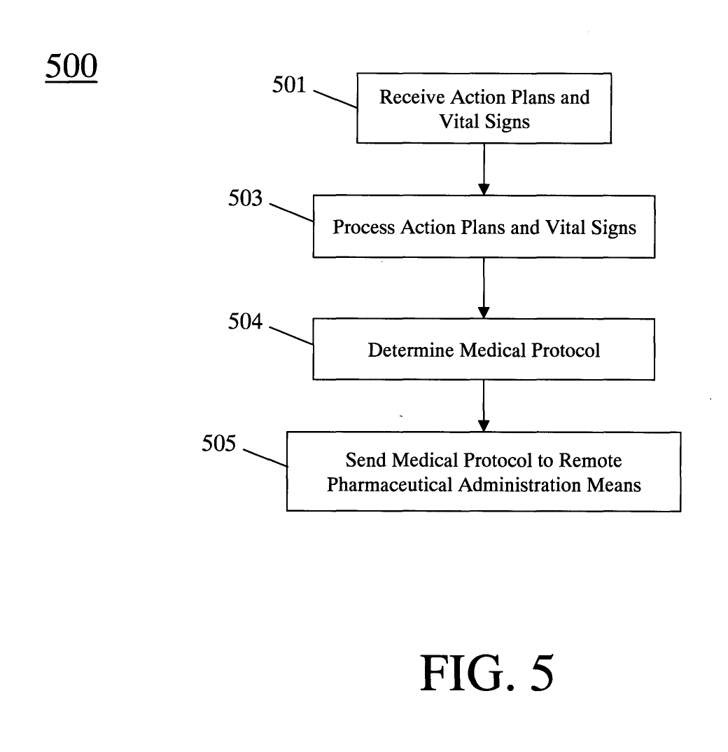

Turning now to FIG. 5, FIG. 5 depicts a flow chart 500 of a method for sending a protocol from pharmaceutical network administration apparatus 300 to remote pharmaceutical administration device 200 in accordance with an exemplary embodiment of the present invention.

Pharmaceutical network administration apparatus 300 receives (501) the action plan and vital signs sent from remote pharmaceutical administration device 200 over wireless communication network 101.

Analysis processor 301 processes (503) the action plan and vital signs. This can include performing calculations to determine whether an alert should be sent to a doctor, whether a phone call should be placed, or whether an alarm should be sent.

Pharmaceutical network administration apparatus 300 determines (504) a medical protocol for the patient. This is preferably done by processing instructions received via means for accepting instructions 305. For example, a doctor can make a medical protocol determination for the patient based upon the vital signs and action plan. By reviewing the vital signs, medical history, recommended action plan, and any other useful information, the doctor can make a determination on what the best course of action is for the patient. This medical protocol is input to pharmaceutical network administration apparatus 300 via means for accepting instructions 305.

Transceiver 307 sends (505) medical protocol to remote pharmaceutical administration device 200 over wireless communication network 101 via transceiver 307.

Returning now to FIG. 4, remote pharmaceutical administration device 200 receives (409) the medical protocol sent by pharmaceutical network administration apparatus 300.

Remote pharmaceutical administration device 200 performs (411) the medical protocol. In an exemplary embodiment, the medical protocol can be to dispense pharmaceuticals 201 to patient 299 via administration means 203. In a further, exemplary embodiment, the medical protocol can be to run further tests on patient 299 via testing apparatus 207, such as an EKG machine, a blood sugar monitor, or a sphygmomanometer. In a further exemplary embodiment, the medical protocol can be to take no action at this time but continue to monitor the vital signs of patient 299.

The present invention thereby provides a method, system, and apparatus that allows for a patient to be diagnosed and treated without requiring all medical personnel to be physically present with the patient.

While this invention has been described in terms of certain examples thereof, it is not intended that it be limited to the above description, but rather only to the extent set forth in the claims that follow.

1. A remote pharmaceutical administration device comprising:
a monitoring device for monitoring the vital signs of a patient;
an analysis processor coupled to the monitoring device for determining an action plan;
a transceiver coupled to the analysis processor for transmitting the vital signs and action plan over a wireless network and receiving a medical protocol over the wireless network for the patient; and
administration means for administering a prescribed medication to the patient based at least in part upon the medical protocol.

2. A remote pharmaceutical administration device in accordance with claim 1, the remote pharmaceutical administration device further comprising pre-stored pharmaceuticals.

3. A remote pharmaceutical administration device in accordance with claim 1, the remote pharmaceutical administration device further comprising a remotely-run testing apparatus.

4. A remote pharmaceutical administration device in accordance with claim 3, wherein the remotely-run testing apparatus comprises an EKG machine.

5. A remote pharmaceutical administration device in accordance with claim 3, wherein the remotely-run testing apparatus comprises a blood sugar monitor.

6. A remote pharmaceutical administration device in accordance with claim 3, wherein the remotely-run testing apparatus comprises a sphygmomanometer.

7. A remote pharmaceutical administration device in accordance with claim 1, wherein the monitoring device comprises a thermometer.

8. A remote pharmaceutical administration device in accordance with claim 1, wherein the analysis processor comprises a local pharmaceutical protocol change.

9. A remote pharmaceutical administration device in accordance with claim 1, wherein the administration means comprises a needle.

10. A remote pharmaceutical administration device in accordance with claim 1, wherein the administration means comprises a gas mask.

11. A remote pharmaceutical administration device in accordance with claim 1, wherein the administration means comprises a pill dispenser.

12. A remote pharmaceutical administration device in accordance with claim 1, the remote pharmaceutical administration device further comprising an input device.

13. A pharmaceutical network administration apparatus comprising:
a network transceiver for receiving an action plan over a wireless network;
a network processor that develops a medical protocol based at least in part upon the action plan; and
wherein the network transceiver transmits the medical protocol over the wireless network.

14. A pharmaceutical network administration apparatus in accordance with claim 13, the pharmaceutical network administration apparatus further comprising means for accepting a pharmaceutical protocol.

15. A pharmaceutical network administration apparatus in accordance with claim 13, wherein the network processor sends an alert message.

16. A method for remotely administering a prescribed medication comprising:
monitoring a vital sign of a patient;
processing the vital signs to determine an action plan for the patient;
determining if a medical specialist should be alerted; and
if a medical specialist should be alerted, sending the action plan and vital signs to a pharmaceutical network administration apparatus via a wireless communication network.

17. A method for remotely administering a prescribed medication in accordance with claim 16, wherein the step of processing the vital signs to determine an action plan for the patient comprises determining a generic action plan, wherein the action plan comprises determining whether the vital sign falls outside of a predetermined range.

18. A method for remotely administering a prescribed medication in accordance with claim 16, the method further comprising the step of determining a medical protocol for the patient.

19. A method for remotely administering a prescribed medication in accordance with claim 18, the method further comprising the step of performing the medical protocol on the patient.

20. A method for remotely administering a prescribed medication in accordance with claim 19, wherein the step of performing the medical protocol on the patient comprises dispensing pharmaceuticals to the patient.
